(19) United States
(12) Patent Application Publication
(10) Pub. No.: US 2026/0166263 A1
(43) Pub. Date: Jun. 18, 2026

(54) ANGIOPLASTY DELIVERY SYSTEM AND METHOD OF MANUFACTURING A DELIVERY SYSTEM

(71) Applicant: B. Braun Melsungen AG, Melsungen (DE)

(72) Inventors: Eberhard Engelien, Wohltorf (DE); Qui Van Huynh, Berlin (DE); Volker Moosig, Berlin (DE); Henrike Reich, Berlin (DE)

(21) Appl. No.: 19/421,575

(22) Filed: Dec. 16, 2025

(30) Foreign Application Priority Data
Dec. 17, 2024 (DE) 10 2024 138 259.7

(51) Int. Cl. A61M 25/00; A61M 25/10

(52) U.S. Cl. CPC A61M 25/002; A61M 25/10; A61M 2207/00; A61M 2209/06

(57) ABSTRACT

A delivery system for percutaneous transluminal angioplasty includes an angioplasty catheter to be sterilized or sterilized. The angioplasty catheter is held on one side on a flat carrier in one or more spiral turns. The angioplasty catheter has a hub at a predetermined proximal end section and at least one balloon at a predetermined distal end section. The carrier has an indentation in which the angioplasty catheter is accommodated. A method for manufacturing a delivery system includes the step of shaping and/or reshaping a flat carrier with an indentation and inserting an angioplasty catheter into the indentation.

### CROSS-REFERENCE TO RELATED APPLICATION

This application claims priority under 35 U.S.C. § 119 to German Application No. 10 2024 138 259.7, filed on Dec. 17, 2024, the content of which is incorporated by reference herein in its entirety.

### FIELD

The disclosure relates to a delivery system for percutaneous transluminal angioplasty with a carrier and an angioplasty catheter held therein, which is to be sterilized or is sterilized, and which has a hub at one end and at least one balloon at the other end. The disclosure also relates to a method for manufacturing a delivery system for percutaneous transluminal angioplasty.

### BACKGROUND

Angioplasty catheters are packaged in sterile packaging, sterilized in it, and then made available. Sterile packaging enables sterilization and also ensures that the catheter remains sterile when stored appropriately until it is ready for use. Recontamination of the sterilized angioplasty catheter after sterilization should generally be ruled out until it is used.

In a manner known from the prior art—such as, for example, in the applicant's product marketed under the registered trademark SeQuent® NEO—angioplasty catheters are held in a spirally wound plastic tube, a so-called “dispenser,” and provided in a sterile bag.

During manufacture, this angioplasty catheter is packaged by threading its tip, which carries the catheter balloon, into the linearly extending dispenser, which is slightly longer than the angioplasty catheter, and pushing it in until the connection or hub of the angioplasty catheter comes into contact with the insertion opening of the dispenser. The inserted angioplasty catheter is then accommodated in the dispenser and protected against damage and kinking. The dispenser is then placed/wound in two to four spiral turns and the spiral turns are secured with clips. The dispenser wound in this way is then placed in a bag, known as a “peel bag,” and the bag is closed, in particular sealed or heat-sealed. The manufacturing process described can only be automated at relatively high cost.

In an alternative manufacturing process, the dispenser is first placed/wound into spiral turns and secured, and then the angioplasty catheter is inserted into the coiled dispenser.

All important information about the angioplasty catheter contained in the package is printed on the peel bag. These include, for example, the catheter type, its item number, batch, date of manufacture, and expiration date. Other relevant data printed on the balloon include the diameter of the guide wire, the nominal diameter and length of the balloon, and the nominal pressure including the maximum permissible pressure (rated burst pressure). In addition, a table is provided showing the pressure-diameter behavior of the balloon, known as the “compliance chart,” with the nominal pressure and maximum permissible pressure highlighted.

When performing percutaneous transluminal angioplasty, the angioplasty catheter is provided to the surgeon by an assistant opening the peel bag. The surgeon removes the dispenser, which is held in a spiral shape by clips, from the sterile interior of the peel bag and pulls the approximately 1.5-to 2-m-long angioplasty catheter out of the dispenser. Finally, the tip of the angioplasty catheter is pulled out of the dispenser, which the surgeon then inserts into the patient via the arterial access sheath.

A disadvantage of the dispenser system is the large amount of plastic required for packaging and supplying the angioplasty catheter. The packaging, i.e., the peel bag and the dispenser, accounts for around 95% of the plastic in the product. Reuse is not possible.

Another disadvantage is that the surgeon must always first completely withdraw the angioplasty catheter before reaching the tip of the catheter in order to flush it and/or thread the catheter onto the guide wire, for example. This means that the angioplasty catheter is already exposed to its full length, even if the task is initially only to thread the catheter tip onto the guide wire and then insert it into the sheath at the patient's access point. The surgeon themself or an assistant must therefore ensure that the meter-long remainder of the withdrawn angioplasty catheter is securely located/held/protected against damage or contamination. In addition, a syringe can also be connected to the hub, which is used to evacuate the catheter, i.e., to create a vacuum (or quasi-vacuum) in the catheter. This makes it even more difficult to handle the catheter safely. In addition to the actual core task of performing angioplasty, this represents an expense and a source of error.

Furthermore, a large number of catheters are or may be used during angioplasty. Examples include the guide catheter for administering contrast medium, several balloon catheters for sequential, step-by-step dilation of a stenosis or for multiple stenoses at a bifurcation, and the respective angioplasty catheter for placing the stent. If each of these catheters is supplied via a dispenser, this results in a considerable amount of plastic that has to be handled in the operating room. In particular, the nominal values and compliance charts printed on the peel bags, or the peel bags themselves, must be kept in a safe place so that the information is always reliably and clearly accessible to the surgeon. In addition, the information should also be provided on the dispenser, as otherwise it will be difficult to identify the angioplasty catheter and determine its specifications once the dispenser containing the catheter has been removed from the peel bag.

### SUMMARY

In contrast, it is the task of the disclosure to avoid or at least mitigate the disadvantages of the prior art. In particular, one task is to create a delivery system for angioplasty that allows the user to remove an angioplasty catheter in a variety of ways. Another task is to create a method for manufacturing such a delivery system that reduces the effort required for automation, reduces the use of resources, and uses more sustainable materials.

A basic idea of the present disclosure is to provide an angioplasty catheter to be sterilized or sterilized in an indentation of a flat carrier. To save space during deployment, the angioplasty catheter is coiled in the indentation, i.e., it is accommodated in one or more spiral turns. The indentation is formed on one side of the flat carrier, meaning that it is accessible from one side. This allows a surgeon or user to remove the carrier and grasp the angioplasty catheter at different points or regions from the aforementioned side, either individually or together, particularly at a hub or at the spiral turns or at a tip of the catheter. This allows the surgeon to access every relevant section of the angioplasty catheter during preparation and performance of the angioplasty, in particular the important end sections of the hub and catheter tip, without having to completely remove/pull out the angioplasty catheter from the carrier, as is the case with the state of the art in dispenser technology.

A delivery system for percutaneous transluminal angioplasty according to the disclosure has a flat carrier and an angioplasty catheter that is to be sterilized or has been sterilized. The angioplasty catheter is held on one side of the flat carrier in one or more spiral turns. The angioplasty catheter has, as described, a proximal end section with a hub or connection and a distal end section, in particular a catheter tip, with at least one catheter balloon. According to the disclosure, the flat carrier has an indentation in which the angioplasty catheter is accommodated, so that the surgeon can safely set down the carrier and pick up the angioplasty catheter at different points, either individually or together, without having to completely remove/pull the angioplasty catheter from the carrier. This creates a delivery system for percutaneous transluminal angioplasty that allows the user to remove the angioplasty catheter in a variety of ways.

According to further embodiment, at least three support points are provided on the carrier at which the catheter is accommodated, held, and/or fixed. The support points may be designed as projections from an edge or edge section of the indentation, as holders, as tabs, as pockets, or as clips.

Preferably, the indentation or the support points relating to the angioplasty catheter are curved inwards, i.e., towards the angioplasty catheter, at least in sections, or are provided with projections directed towards the center, in order to prevent the angioplasty catheter from slipping out of the indentation or out of the support points.

According to further embodiment, the angioplasty catheter is accommodated and held on the carrier in such a way that it can be removed by grasping the hub and pulling it out, and/or that it can be removed in one piece, and/or that the hub and/or the balloon can be removed separately.

Each of the removal variants offers the surgeon controlled handling of the angioplasty catheter, in particular of the hub or proximal end section and/or the balloon or distal end section.

In the case of removal as a whole, the user or operator can remove the angioplasty catheter in a coiled state, then connect a syringe for evacuation, for example, thread the distal end section/tip/balloon onto a guide wire, and then uncoil the angioplasty catheter and lay it down straight. During this handling of the coiled angioplasty catheter, the user has better control over the entire angioplasty catheter than if they first stretch and set down the angioplasty catheter and then connects the syringe and thread the distal end section/tip/balloon onto the guide wire.

According to a preferred embodiment, one or more spiral turns of the angioplasty catheter exhibit flexural elasticity.

Preferably, the catheter should not be stored in a coiled state, as its elasticity would cause it to stretch uncontrollably, which could render it non-sterile.

According to a preferred embodiment, the indentation has an edge or at least an edge section at which the one or more spiral turns are radially supported on the outside in a flexurally elastic manner. In this way, the angioplasty catheter is tensioned in the indentation by its own tension or bending stress and is thus held on the carrier without the need for additional support points or retaining devices, such as brackets, tabs, or clips.

According to an advantageous further development, the indentation has at least one separate indentation section or accommodating section in which one of the end sections, i.e., the hub or the catheter tip, is accommodated. This separate indentation section or accommodating section enables improved retention of the respective end section on the carrier, but also more precise gripping of the respective end section by the operator.

According to a further development, the indentation with reference to the edge section, in particular with reference to the one or more spiral turns, has a first accommodating section that tapers off approximately tangentially, in which the hub is accommodated.

Preferably, the first accommodating section is designed with an offset to an outer contour and/or outer surface of the hub, so that the hub is accommodated and firmly anchored therein in a predetermined or defined position and can no longer slip out unintentionally.

According to a further development, the indentation has a second accommodating section that tapers tangentially in relation to the one spiral turn or one of the several spiral turns, in which the distal end section is accommodated.

According to a further advantageous embodiment of the present disclosure, the first accommodating section and/or the second accommodating section of the carrier are designed to be bendable so that the relevant accommodating section can be bent or folded away from the hub or the distal end section of the catheter in such a way that the hub and/or the distal end section/balloon are exposed and can be easily grasped.

To form a defined crease line or fold line, a continuous or interrupted weak point or impression may be provided on the carrier. Preferably, the weak point, crease line, fold line, or impression is linear.

If the carrier is not part of the sterile barrier, continuous or sectional perforation of the carrier may be provided to form the weak point, crease line, or fold line.

According to a further embodiment, the weak point or impression reduces the thickness of the carrier, so that it has a lower moment of inertia in the region of the weak point or impression and can be bent or folded more easily at this point.

In a further development, an edge is provided around the outer circumference of the carrier, in particular to achieve a basic rigidity of the carrier against buckling.

In the case of a further development with an edge running around the outside of the carrier and with a bend line or fold line, this edge is preferably interrupted or recessed in the region of the bend line or fold line. This ensures that the fundamental stiffening effect of this edge is specifically interrupted in the region of the crease line or fold line, allowing for easy creasing or folding.

Alternatively, instead of the crease line or fold line, a separation line may be provided along which the corresponding part of the carrier can be separated, for example torn off. However, since the torn-off part must be disposed of separately, which creates additional work, this is not the preferred method.

According to a further development, the at least one weak point, impression, crease line, fold line, or perforation divides the carrier into a first segment having the first accommodating section and/or a second segment having the second accommodating section and a third segment having the spiral turns.

According to a further embodiment, the indentation has an outer contour that borders the angioplasty catheter accommodated therein, at least in sections. To ensure that the angioplasty catheter can be easily inserted and removed, an offset of the outer contour of the indentation relative to the angioplasty catheter is preferably provided.

Preferably, the outer contour of the indentation borders the angioplasty catheter along its entire length, so that the entire angioplasty catheter is held/fixed in the indentation.

In a simple manufacturing process, which makes it easy and highly automatable to insert the angioplasty catheter into the indentation of the carrier, particularly during the manufacture of the delivery system, the carrier forms a shell with its edge or edge section.

According to a preferred further development, the edge or edge section at which the one or more spiral turns are radially supported on the outside in a flexurally elastic manner only wraps around once at most, so that a shell is formed by the carrier. The spiral turns can thus be supported together as a bundle or group radially on the outside at the edge or edge section. This has the advantage that the angioplasty catheter with its spiral turns can be grasped “as a whole” in the simplest possible way, or conversely, can be inserted “as a whole” into the indentation when the delivery system is being manufactured.

In another further development, the edge or edge section at which one or more spiral turns are radially supported on the outside in a flexurally elastic manner runs around multiple times, in particular in a spiral shape. This means that the spiral turns are individually supported. This means that one spiral turn is supported at each circumference of the edge or edge section. You could also say that the angioplasty catheter is radially supported on the outside, spiral turn by spiral turn.

In one possible design, the spirally circumferential edge section is formed by a radially outer flank of a spirally circumferential groove.

According to a preferred further development, the edge section at which the one or more spiral turns are radially supported on the outside in a flexibly elastic manner is, at least in sections, designed to be so steep and/or undercut that when the angioplasty catheter is gripped at the hub and the angioplasty catheter is pulled out of the indentation, the angioplasty catheter is guided at the edge section in such a way that the spiral turn or spiral turns are prevented from popping out of the indentation.

Such a design allows—in addition to or as an alternative to the above-described gripping of the angioplasty catheter “as a whole”—the guided withdrawal of the angioplasty catheter from the carrier, as the surgeon is accustomed to doing, for example, with the state-of-the-art spiral-wound, tubular dispenser.

The undercut may be formed completely around the circumference, in particular by a completely circumferential projection.

Alternatively or additionally, the undercut may be formed in sections around the circumference, in particular by several projections.

Both possible forms of the undercut—completely circumferential or sectional circumferential—prevent the angioplasty catheter from accidentally popping out when it is completely resting in the indentation.

The undercut, which runs around in sections, also has the advantage that when the hub or tip of the catheter is removed from the indentation, the rest of the catheter only slides or jumps out of the indentation until it comes into contact with a leading edge of the next section of the undercut, in particular the next projection that forms this undercut. At this edge, it is then stopped by its outward bending elasticity, which acts as a self-locking mechanism. For further removal, it must be deformed inward again, contrary to its outward bending elasticity, in order to overcome this projection. Due to its bending elasticity, this cannot happen automatically, which prevents the angioplasty catheter from popping out unintentionally. Instead, manual intervention by the user or operator is required to guide the angioplasty catheter around the projection and continue the extraction in a controlled manner until the next projection. Here, manual intervention is required again, and so on.

According to a preferred embodiment, at least three projections are provided at the radially outer edge of the indentation where the spiral turn or spiral turns of the angioplasty catheter are supported, for each spiral turn of the angioplasty catheter. The projections are distributed over a circumferential angle of the respective spiral turn, wherein the circumferential angle is greater than 180° so that the self-locking described above against uncontrolled popping out or slipping of the angioplasty catheter is achieved.

A combination of the completely circumferential and the sectional circumferential undercut (projections) is possible, for example, by having the projections extend repeatedly radially inward from the completely circumferential undercut.

In order to improve the grip of the angioplasty catheter in the indentation, according to a preferred further development, a gripping recess is provided on the carrier, in particular in an edge region of the indentation, in particular in a region of the hub and/or the spiral turn or spiral turns and/or the catheter balloon, which is at least partially spanned by the accommodated angioplasty catheter, in particular by the hub, the spiral turn or spiral turns or the catheter balloon.

Alternatively or additionally, at least one through recess is provided on the carrier, which is spanned at least in sections by the accommodated angioplasty catheter, in particular by the hub, by the spiral turn or spiral turns, or by the catheter balloon. A through recess is understood to be a hole in the carrier.

Alternatively, one could say that the indentation is interrupted by the gripping recess or the through recess. In the case of the gripping recess, interrupted means that the gripping recess is deeper than the indentation.

In this way, the gripping recess or the through recess of the hub, the one or more spiral turns, or the end section with the balloon can be easily gripped/under-gripped/or grasped and lifted or removed from the indentation and/or pulled off.

Preferably, two, three, or four gripping recesses or through recesses are provided distributed along one or more of the spiral turns, preferably at equal angular intervals, in order to improve the above-mentioned removal of the angioplasty catheter “as a whole.”

According to a preferred embodiment, the carrier is designed and constructed to be permeable to a sterilization medium and impermeable to germs. Of course, this further development is not effective in the above-mentioned embodiment, which has a through recess to improve the gripability of the angioplasty catheter.

Preferably, the carrier is permeable to water vapor and/or ethylene oxide gas, which, in the case of the design with a gripping recess, can preferably be achieved by the porosity of the material used. In the case of the design with the above-mentioned through recess to improve grip, such porosity is not necessary.

According to a preferred embodiment, the carrier is thermoformed from a film, preferably a biofilm, or is molded from a fiber material, preferably a biofiber material. By using the aforementioned organic material, the carrier completely avoids plastic and the amount of plastic waste produced is reduced. Sustainability is thus improved.

According to a further development, the carrier has at least one notch, thereby reducing the amount of material used and the weight of the carrier.

Preferably, the notch is provided centrally on the carrier, in particular radially inward or concentrically with respect to the one or more spiral turns.

According to a further development, the indentation is spanned by a sealing film connected to the carrier, so that the angioplasty catheter is held in the indentation by the sealing film, i.e., it is secured against unintentional falling out or popping out.

According to a preferred embodiment, the indentation is sealed germ-tight with the sealing film. In this case, the carrier forms a sterile barrier with the sealing film. A prerequisite for this further embodiment is that the carrier does not have any of the above-described through recesses to improve gripability.

According to an alternative embodiment, a packaging bag or “peel bag” is provided in which the carrier is arranged unsealed, the packaging bag being sealed in a germ-tight manner. In this case, the carrier therefore only serves to hold and subsequently store/provide the angioplasty catheter, whereas the sterile barrier is formed by the packaging bag or peel bag.

This further embodiment with a closure bag for forming the sterile barrier is particularly necessary if the carrier has at least one of the through recesses described above for improved gripping of the angioplasty catheter.

According to a preferred embodiment, at least one parameter of the angioplasty catheter, for example the catheter type, its item number, its batch, its date of manufacture, its expiry date, the diameter of the guide wire, the nominal diameter and length of the balloon, the nominal pressure, the maximum permissible pressure, and/or the pressure-diameter behavior of the balloon, is depicted on the sealing film.

To ensure that the sealing film remains on the carrier and, in particular, that the above-mentioned information on at least one parameter is not lost when the sealing film is opened, an opening section of the sealing film is detachably connected to the carrier, whereas a connecting section of the sealing film is connected to the carrier more firmly than the opening section, in particular to remain on the carrier. When the delivery system is open, the angioplasty catheter lies openly in the indentation of the carrier, and the characteristic values are still secured against loss on the carrier. This means that a specification of the angioplasty catheter is always present.

The angioplasty catheter can then be prepared for angioplasty after partially removing the sealing film, without having to be removed. After partially removing the sealing film, the carrier can also be reused for temporary storage of the angioplasty catheter.

A method for manufacturing a delivery system for percutaneous transluminal angioplasty, in particular for manufacturing a delivery system designed in accordance with at least one aspect of the preceding description, comprises the following steps:
shaping or reshaping of a flat carrier with an indentation; and
inserting an angioplasty catheter, which has a hub at a predetermined proximal end section and at least one catheter balloon at a predetermined distal end section, into the indentation in such a way that the angioplasty catheter is accommodated and held in the indentation in one or more spiral turns.

The flat carrier with the indentation can be manufactured at low cost by means of shaping or reshaping. Shaping or reshaping also allows for almost unlimited variation in the shape of the indentation. This allows the indentation to be produced in a cost-effective manner so that the angioplasty catheter can be inserted into the indentation in the simplest possible way and held securely there. This creates a method for manufacturing the delivery system that allows the effort required for automation to be kept low or reduced.

According to a further development of the method, the reshaping is carried out by deep drawing a film, preferably a biofilm, and the shaping is carried out by fiber casting a fiber material, preferably a biofiber material. This increases sustainability. Non-fiber materials can be injection molded.

In one variant of the method, the following steps are provided for:
spanning the indentation of the carrier with a sealing film; and
connecting the sealing film to the carrier, for example by sealing (heat sealing), gluing, fusion welding (including ultrasonic welding, laser welding, and heating element butt welding), or press welding. In this way, the angioplasty catheter is packaged in a germ-tight manner in a receptacle bounded by the carrier and the sealing film. The carrier thus forms the sterile barrier together with the sealing film.

As an alternative or supplement to the steps of spanning the indentation of the carrier with a sealing film and connecting the sealing film to the carrier, the method comprises the following steps:
packing the carrier in a closure bag; and
sealing the closure bag, for example using one of the connection techniques mentioned in the previous paragraph. In this way, the carrier and the catheter attached to it are packaged in a germ-tight manner in the closure bag without the indentation having to be sealed separately with a sealing film.

These last two steps are particularly necessary if the carrier is not at all suitable for forming a sterile barrier. This is the case, for example, if it has at least one of the through recesses described above for improved gripping of the angioplasty catheter.

### BRIEF DESCRIPTION OF THE DRAWINGS

The disclosure is further explained below with the aid of drawings.

FIG. 1 shows a top view of a first embodiment of a delivery system according to the disclosure, with a carrier, an angioplasty catheter accommodated on the carrier, and a partially removed sealing film;

FIG. 2 shows a rear view of the delivery system according to FIG. 1;

FIG. 3 shows a top view of a second embodiment of a delivery system according to the disclosure, with a carrier and an angioplasty catheter accommodated on the carrier with the sealing film completely removed;

FIG. 4 shows a rear view of a third embodiment of a delivery system according to the disclosure, comprising a carrier, an angioplasty catheter accommodated on the carrier, and a sealing film;

FIG. 5 shows a cross-section of the delivery system according to FIG. 1;

FIG. 6 shows a cross-section of a fourth embodiment of a delivery system according to the disclosure;

FIG. 7 shows a cross-section of a fifth embodiment of a delivery system according to the disclosure; and

FIG. 8 shows a flowchart of a method for manufacturing a delivery system according to an embodiment.

### DETAILED DESCRIPTION

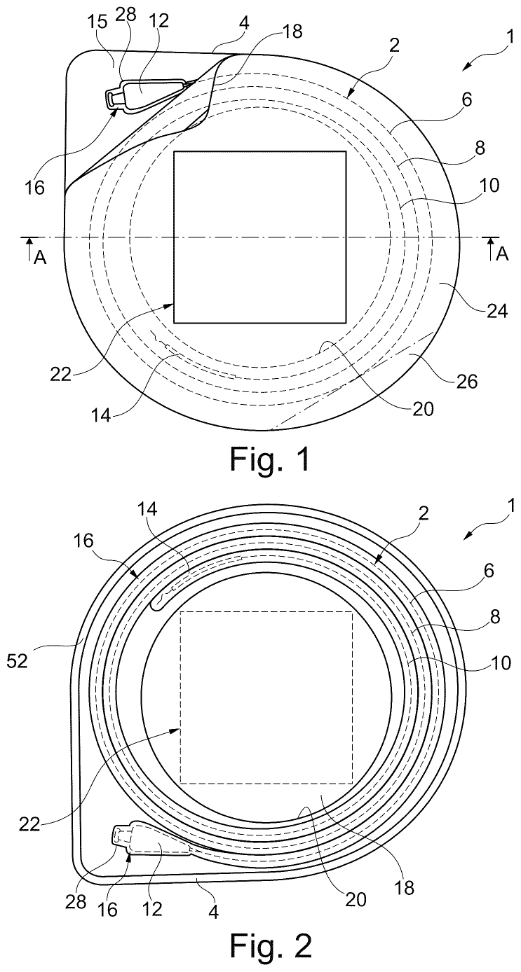

FIG. 1 shows a delivery system 1 for percutaneous transluminal angioplasty according to a first embodiment in a top view as it appears to a surgeon when opening the delivery system 1.

The delivery system 1 has a predominantly circular, flat carrier 4, on which a sterile angioplasty catheter 2 is arranged and held on one side. The angioplasty catheter 2 is held rolled up on the carrier 4 in several spiral turns 6, 8, 10. At its predetermined proximal end section, the angioplasty catheter 2 has a hub 12 or connection, in particular for connecting an angioplasty syringe, and at its predetermined distal end section it has a catheter balloon 14.

The carrier 4 has an indentation 16 on its flat side 15, in which the complete angioplasty catheter 2 is accommodated and held. The indentation 16 has a first accommodating section 28 or retaining section, which extends tangentially in relation to the radially outermost spiral turn 6 and in which the hub 12 is accommodated. The first accommodating section 28 has an offset to an outer contour of the hub 12, which is dimensioned so that the hub 12 can be easily inserted into the first accommodating section 28, is held in place to prevent it from slipping or sliding out, and can still be easily gripped and removed. In the region of the first accommodating section 28, the carrier 4 has a corner section that interrupts its circular shape.

According to FIG. 1, indentation 16 is spanned by a sealing film 18. The sealing film 18 is connected to the flat side 15 of the carrier 4 in a material-locking manner, in particular by heat sealing.

The indentation 16 shown in FIG. 1 is designed to be spiral-shaped, wherein the illustration in FIG. 1 does not show the spiral arms of the indentation 16 that are covered by the sealing film 18 in order to avoid overloading FIG. 1. The spiral turns 6, 8, 10 run in a spiral indentation 16 from the hub 12 to the catheter balloon 14. Both the spiral turns 6, 8, 10 and the catheter balloon 14 are shown in dashed lines in FIG. 1, as they are covered by the sealing film 18.

In the illustration shown in FIG. 1, the sealing film 18 has been pulled back from the flat side 15 of the carrier 4 just far enough to expose the hub 12, which is intended to represent the moment when the delivery system 1 is opened by the surgeon for removal of the angioplasty catheter 2 in preparation for or during the angioplasty procedure.

When the delivery system 1 is closed, the sealing film 18 is connected to the carrier 4 over its entire length or across its entire surface on the flat side 15. The sealing film 18 then forms, together with the carrier 4, the sterile barrier for the angioplasty catheter 2 held in the indentation 16. The indentation 16 represents a sterile accommodating space for the angioplasty catheter 2.

The carrier 4 and the sealing film 18 of the delivery system 1 according to FIG. 1 thus form a compact sterile barrier for the angioplasty catheter 2 with minimal use of material.

A data field 22 containing all relevant parameters of the angioplasty catheter 2 is displayed on the upper side or outer side of the sealing film 18 facing the surgeon. The parameters are the type of catheter included, its item number and batch, its date of manufacture and expiry date, the diameter of the guide wire, the nominal diameter and length of the balloon, the nominal pressure and maximum permissible pressure, and the pressure-diameter behavior of the balloon, known as the compliance chart.

In a region diametrically opposite the corner region of the flat side 15, i.e., diametrically opposite the first accommodating section 28 or hub 12, the sealing film 18 has a connecting section 26 that is firmly bonded to the carrier 4 in such a way that the above-mentioned parameters and information about the angioplasty catheter 2 are not lost and remain available even when the sealing film 18 is opened. An opening section 24 of the sealing film 18, which is provided diametrically opposite the connecting section 26, extends from the connecting section 26 to the corner region of the flat side 15, as shown in FIG. 1.

The opening section 24 is less firmly connected to the carrier 4 than the connecting section 26 so that it can be removed by the operator with reasonable effort.

The opening section 24 is dimensioned so that it spans the entire angioplasty catheter 2, so that when the opening section 24 is pulled out, the hub 12, all spiral turns 6, 8, 10, and the catheter tip 14 are exposed until the more firmly connected connecting section 26 makes removal suddenly more difficult and the surgeon can intuitively stop the removal.

The aforementioned, more secure connection of the connecting section 26 is only designed to be secure enough that the connecting section 26 can be removed manually or automatically for subsequent waste separation.

The angioplasty catheter 2 can then be prepared for angioplasty up to the connecting section 26 after partial removal of the sealing film 18, without having to be removed from the carrier 4, or more precisely from its indentation 16. After partial removal of the sealing film 18, the carrier 4 can also be reused for temporary storage of the angioplasty catheter 2.

The carrier 4 has a circular through recess or through notch 20 in the center, i.e., approximately concentric with the spiral turns 6, 8, 10, in order to keep the material consumption for the delivery system 1 low.

FIG. 2 shows a rear view, or bottom view, of the delivery system 1 according to FIG. 1. The flat character of the carrier 4 is clearly visible in that the indentation 16 described above in the flat side 15 of the carrier 4 bulges toward the observer. As described above, the angioplasty catheter 2 (shown as a dashed line) extends from the first accommodating section 28 of the indentation 16 with its radially outer spiral turn 6, its radially middle spiral turn 8, and its radially inner spiral turn 10 to its distal end section with the balloon catheter 14.

According to the first exemplary embodiment shown in FIGS. 1 and 2, the indentation 16 extends from the first accommodating section 28 as a spiral groove.

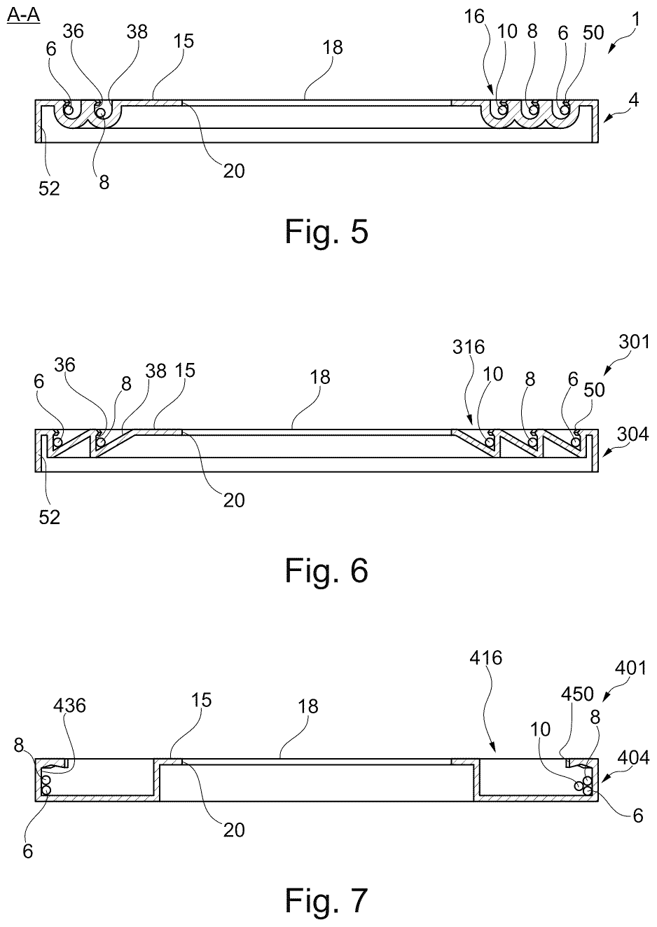

To protect against bending, the carrier 4 has a rear edge 52 running around it (see also FIG. 5). The edge 52 is formed at the rear so as not to impede the closure or sealing of the front indentation 16 with the sealing film 18 and the removal of the angioplasty catheter 2 from the indentation 16.

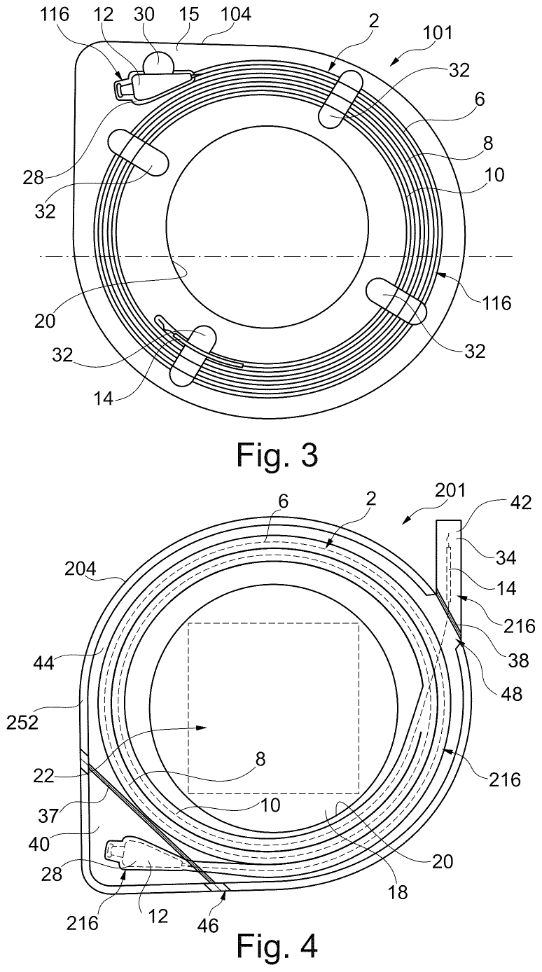

FIG. 3 shows a top view of a second embodiment of a delivery system 101 according to the disclosure, with a carrier 104 and the angioplasty catheter 2 held on the carrier 104 with the sealing film completely removed.

In order to avoid overloading the description, only the differences from the first embodiment according to FIGS. 1 and 2 will be discussed below. Components that are identical across the different embodiments are also marked with the same reference symbols.

According to FIG. 3, several gripping recesses 30, 32 are provided on the carrier 104, more precisely on its flat side 15, each of which opens into the indentation 116. The gripping recess 30 opens laterally into the first accommodating section 28, so that the hub 12 can be gripped laterally with a finger and removed from the first accommodating section 28. The other four gripping recesses 32 are arranged at equal angular intervals along the spiral turns 6, 8, 10, wherein they are formed deeper in the flat side 15 than the spiral-shaped indentation 116 and thus interrupt the indentation 116. Thus, the spiral turns 6, 8, 10 span the respective gripping recess 32. This allows the spiral turns 6, 8, 10 to be gripped together from the radial inside and the radial outside, and the angioplasty catheter 2 can be removed “in one piece” or “as a whole” from the carrier 104.

In an alternative embodiment of a disclosure-compliant delivery system (not shown), access to the angioplasty catheter is achieved by means of at least one through recess, which is essentially a hole in the carrier, rather than by means of gripping recesses. It is important to note that the perforated carrier can no longer contribute to a sterile barrier. It is then limited solely to its carrying and holding function. In this embodiment, additional packaging is then necessary to form the sterile barrier, for example, by placing the carrier with the angioplasty catheter held thereto in a closure bag (“peel bag”) that is impermeable to germs.

FIG. 4 shows a rear view of a third embodiment of a delivery system 201 according to the disclosure, with a carrier 204, an angioplasty catheter 2 accommodated on the carrier 204, and a sealing film 18.

In a region of the carrier 204 that is arranged approximately diametrically opposite the first accommodating section 28, the indentation 216 on the carrier 204 forms an elongated protrusion/elongated extension and thus a second accommodating section 34 in which the catheter balloon 14 is separately accessible from the remaining spiral turns 6, 8, 10 and can thus be grasped separately when the sealing film 18 is removed. In this way, the catheter tip/catheter balloon 14 can be grasped separately by the surgeon and inserted into the sheath at the patient's arterial access in preparation for or during angioplasty without having to remove the remaining components of the angioplasty catheter 2, i.e., the spiral turns 6, 8, 10, and the hub 12. This allows the majority and longest part of the angioplasty catheter 2 to remain neatly and securely stored in the carrier 204 during insertion of the catheter tip, which makes handling easier for the surgeon.

To make it even easier for the surgeon to grasp the hub 12 and the catheter tip/catheter balloon 14, two linear weak points 37, 38, each formed by an impression, are provided on the carrier 204, which serve as a bend or fold line. This weakens the moment of inertia of the carrier 204 to such an extent that its buckling/folding resistance is reduced.

These impressions 37, 38 divide the carrier 204 into three segments 40, 42, and 44 that can be bent/folded away relative to each other. This allows the first accommodating section 28 and the second accommodating section 34 to be bent away from the central third segment of the carrier 204 as desired, thereby exposing the hub 12 and/or the catheter balloon 14 as desired and allowing them to be grasped completely unhindered.

In the exemplary embodiment shown in FIG. 4, the impressions 37, 38 run linearly. Alternatively, they can be curved either completely or in sections, for example, adapted to the curved course of the indentation 216 in the region of the respective adjacent spiral turn 8 or 6.

The carrier 204 of the exemplary embodiment shown in FIG. 4 also has an edge 252 running around its outer circumference. In contrast to edge 52 of the exemplary embodiment shown in FIGS. 1 and 2, edge 252 shown in FIG. 4 is interrupted in the regions of impressions 37 and 38. The edge 252 has a notch 46, 48 in each of these regions so that its stiffening effect is specifically interrupted there. This allows and facilitates the bending or folding away of the respective segment 40, 42 from the central, third segment 44, wherein the edge 252, apart from the notches 46, 48, continues to fulfill its stiffening function.

FIG. 5 shows a section A-A of the delivery system 1 according to FIG. 1. Accordingly, the spiral-shaped circumferential indentation 16 in FIG. 5 is cut on the right in the region of the three spiral turns 6, 8, 10 and in FIG. 5 on the left only in the region of the radially outer spiral turn 6 and the radially middle spiral turn 8. The indentation 16 is designed as a groove with a rounded groove base.

Both flanks or edges of the groove, i.e., a radially outer edge or edge section 36 of the indentation 16 and a radially inner edge or edge section 38 of the indentation 16, are formed so steeply in relation to the flat side 15 that the groove/indentation 16 tapers slightly again toward the flat side 15. In this way, a completely circumferential undercut is formed on the indentation 16, in particular from the radially outer edge or edge section 36, through which the spiral turns 6, 8, 10 are fundamentally secured against popping out or slipping out of the groove/indentation 16.

In addition to the completely circumferential undercut, a partial circumferential undercut is provided, which is formed by isolated projections 50 that are arranged at regular intervals along the radially outer edge or edge section 36. In the exemplary embodiment shown, four and three such projections 50 are provided per spiral turn 6, 8, and per spiral turn 10, respectively, of which only two per spiral turn 6, 8 and only one per spiral turn 10 are visible due to the sectional view in FIG. 5. The projections 50 are arranged substantially evenly distributed over the circumferential angle of the respective spiral turn 6, 8, 10.

If, for example, the user removes balloon 14 of angioplasty catheter 2 from indentation 16 as shown in FIG. 1, the above-mentioned complete circumferential undercut of edge 36 is overcome by this removal alone, and catheter 2 begins to slide along this undercut due to its outward bending elasticity. The angioplasty catheter 2 slides out of the indentation 16 only until it comes into contact with the leading edge of one of the projections 50. Here too, the bending elasticity acts radially outward, causing the angioplasty catheter 2 to self-lock at the relevant projection 50. If the user now deforms (in the sense of elastic deformation) the angioplasty catheter 2 back radially inward at this point, the relevant projection 50 can be overcome and the removal can be continued in a controlled manner until the next projection 50, and so on. The term “deforming” here means that the part of the catheter protruding from the carrier is moved toward the center of the carrier, wherein the catheter is partially elastically deformed.

Another advantage of the completely circumferential undercut and the projections 50 is that the angioplasty catheter 2 can be pulled out of the carrier 4 at the hub 12 (see FIG. 1) in a direction approximately tangential to the radially outer spiral turn 6 in such a way that it remains guided in the groove/indentation 16 until the end of the withdrawal process and does not jump out of the groove/indentation 16 unintentionally.

FIG. 6 shows a cross-section of a fourth embodiment of a delivery system 301 according to the disclosure. In contrast to the embodiment described above in FIGS. 1, 2, and 5, the indentation 316 is designed as a groove with a highly asymmetrical cross-section. The radially outer edge section 36 of the indentation 316 is designed in the same way as in FIG. 5, i.e., steep and with the aforementioned completely circumferential undercut, as well as with the projections 50. A radially inner edge section 38 of the indentation 316, or the radially inner flank of the groove 316, is flat in comparison. In this way, when manufacturing the delivery system 301 shown in FIG. 6, the angioplasty catheter 2 can be more easily inserted into the indentation 316, either manually or automatically.

Due to the elasticity of the spiral turns 6, 8, 10, the angioplasty catheter 2, as in the previously described embodiments, is tensioned radially outward against the edge section 36 and radially supported there. This support, together with the aforementioned completely circumferential undercut and the projections 50 on the radially outer edge section 36, also means that in the embodiment shown in FIG. 6, the angioplasty catheter 2 is protected against popping out when it is pulled out of the indentation 316. The effect and advantages of the projections 50 are the same in the delivery system 301 as in the delivery system 1 according to FIGS. 1, 2, 5.

FIG. 7 shows a cross-section of a fifth embodiment of a delivery system 401 according to the disclosure. In contrast to all of the aforementioned embodiments, the indentation 416 is not designed to run spirally around, but rather runs around a maximum of once. Apart from the corner region described above, it is designed in a circular ring shape so that all spiral turns 6, 8, 10 are supported together, as a “group” or “bundle,” so to speak, at the radially outer edge section 436. The indentation 416 is thus very wide in the radial direction, which further simplifies the insertion of the angioplasty catheter 2, whether manually or automatically, during the manufacture of the delivery system 401. In this embodiment, too, the radially outer edge section 436 is designed with the aforementioned full-circumference undercut and with projections 450 to enable the aforementioned removal of the angioplasty catheter 2 without it popping out.

Compared to the projections 50 shown in FIGS. 5 and 6, the projections 252 shown in FIG. 7 are shaped like hooks, claws, or rakes and extend significantly further and with more undercut radially inward into the indentation 416. This is due to the fact that the spiral turns 6, 8, 10 arranged in a cluster are not individually guided, as is the case, for example, with the individually and spirally guided spiral turns 6, 8, 10 shown in FIGS. 1 through 6. When pulling or lifting, for example, the hub 12 out of the indentation 416, the spiral turns 6, 8, 10 may therefore inhibit and carry each other to a small extent. The widely protruding projections/rakes 252 then prevent, for example, one spiral turn from unintentionally lifting the other and pulling it out of the indentation 416.

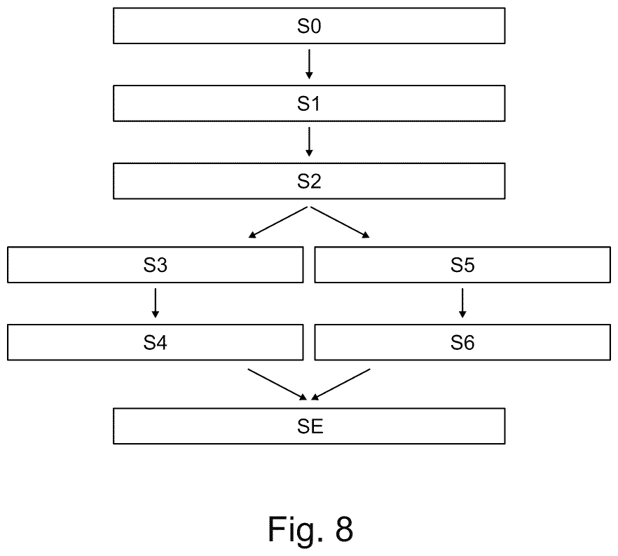

FIG. 8 shows a flowchart of a method according to the disclosure for manufacturing a delivery system according to the disclosure, in particular a delivery system designed in accordance with at least one aspect of the preceding description.

This initially involves step S0 Initialize or Start manufacture. The further description is provided for illustrative purposes with reference to FIGS. 1, 2, and 5, which show the first embodiment of the manufactured delivery system 1.

Step S0 Initialization is followed by step S1 Shaping and/or reshaping of the flat carrier 4 with indentation 16. The shaping and/or reshaping can be carried out by deep drawing a film, preferably a bio-film, or by fiber casting a fiber material, preferably a bio-fiber material. Such bio-films or bio-materials increase sustainability. Injection molding can be performed with non-fiber materials.

This is followed by the manual or, preferably, automated insertion S2 of the angioplasty catheter 2 into the indentation 16 in such a way that the angioplasty catheter 2 is accommodated and held in the indentation 16 in several spiral turns 6, 8, 10. The retention results, on the one hand, from the shape of the indentation 16, which, as described above, has the radially outer edge section 36 with undercut and the first accommodating section 28, and, on the other hand, from the elasticity of the spiral turns 6, 8, 10, due to which the angioplasty catheter 2 is flexibly tensioned against the radially outer edge section 36.

In order to package the angioplasty catheter 2 in a sterile manner and to form a sterile barrier, a variant of the method includes the steps of spanning S3 the indentation 16 with a sealing film 18 and connecting S4 the sealing film 18 to the carrier 4, in particular by means of adhesive bonding or ultrasonic welding, so that the angioplasty catheter 2 is packaged in a germ-tight manner in the receptacle bounded by the carrier 4 and the sealing film 18.

As an alternative or supplement to steps S3 and S4, step S5 of packing the carrier in a closure bag and step S6 of sealing the closure bag may be performed so that the carrier and the catheter attached to it are packed in the closure bag in a germ-tight manner.

This is followed by step SE End of manufacture.

### LIST OF REFERENCE SIGNS

1 Delivery system
2 Angioplasty catheter
4; 104; 204; 304; 404 Carrier
6, 8, 10 Spiral turns
12 Hub
14 Catheter balloon
15 Flat side
16; 116; 216; 316; 416 Indentation
18 Sealing film
20 Through notch
22 Data field
24 Opening section
26 Connection section
28 First accommodating section
30, 32 Gripping recess
34 Second accommodating section
36 Radial outer edge section
37 First impression
38 Second impression
40 First segment
42 Second segment
44 Third segment
46 First notch
48 Second notch
50; 450 Undercut/projection
52; 252 Edge
S0 Step Start Manufacture
S1 Step Shaping the carrier
S2 Step Inserting the angioplasty catheter
S3 Step Spanning the carrier with sealing film
S4 Step Connecting
S5 Step Packing the carrier in closure bag
S6 Step Sealing the closure bags
SE Step End Manufacture

1. A delivery system for percutaneous transluminal angioplasty, the delivery system comprising:
an angioplasty catheter configured to be sterilized; and
a carrier,
the angioplasty catheter being held on one side of the carrier in one or more spiral turns,
the angioplasty catheter comprising a proximal end section with a hub and a distal end section with at least one balloon, and
the carrier comprising an indentation in which the angioplasty catheter is accommodated.

2. The delivery system according to claim 1, wherein:
the one or more spiral turns of the angioplasty catheter has a bending elasticity, and
the indentation has an edge section at which the one or more spiral turns are radially outwardly flexibly supported due to the bending elasticity.

3. The delivery system according to claim 2, wherein:
the indentation has a first accommodating section or retaining section that extends tangentially relative to the edge section, and
the hub is accommodated in the first accommodating section or retaining section.

4. The delivery system according to claim 3, wherein the first accommodating section or retaining section extends tangentially relative to the one or more spiral turns.

5. The delivery system according to claim 3, wherein the first accommodating section or retaining section is formed with an offset to an outer contour of the hub.

6. The delivery system according to claim 3, wherein:
the indentation has a second accommodating section or retaining section that extends tangentially relative to at least one of the one or more spiral turns, and
the distal end section of the angioplasty catheter, or at least the at least one balloon, is accommodated in the second accommodating section or retaining section.

7. The delivery system according to claim 6, wherein the first accommodating section is bendable away from the proximal end section or hub in such a way that the proximal end section or hub is exposed and easily graspable.

8. The delivery system according to claim 6, wherein the second accommodating section is bendable or foldable away from the distal end section or balloon in such a way that the distal end section or balloon is exposed and easily graspable.

9. The delivery system according to claim 2, wherein the edge section circulates at most once and the one or more spiral turns comprise a plurality of spiral turns that are supported at the edge section as a bundle or cluster.

10. The delivery system according to claim 2, wherein the edge section runs around multiple times, and the angioplasty catheter is supported on the edge section spiral turn by spiral turn.

11. The delivery system according to claim 2, wherein the edge section is designed to be steep and/or undercut at least in sections in such a way that when the angioplasty catheter is gripped at the hub and the angioplasty catheter is pulled out of the indentation, the angioplasty catheter is guided at the edge section in such a way that the one or more spiral turns are prevented from popping out of the indentation.

12. The delivery system according to claim 1, wherein the carrier comprises at least one gripping recess or at least one passage recess on the carrier, the at least one gripping recess or at least one passage recess being spanned at least in sections by the angioplasty catheter.

13. The delivery system according to claim 1, wherein the carrier is permeable to a sterilization medium and impermeable to germs.

14. The delivery system according to claim 1, wherein the carrier is made of film or a fiber material.

15. The delivery system according to claim 1, wherein:
the indentation is spanned by a sealing film connected to the carrier so that the indentation is sealed in a germ-tight manner by the sealing film, or
a packaging bag is provided in which the carrier is arranged, the packaging bag being sealed in a germ-tight manner.

16. The delivery system according to claim 1, wherein the indentation is spanned by a sealing film connected to the carrier so that the indentation is sealed in a germ-tight manner by the sealing film, the delivery system further comprising at least one of:
at least one characteristic value or parameter of the angioplasty catheter depicted on the sealing film;
an opening section in the sealing film that is detachably connected to the carrier; or
a connecting section of the sealing film that is connected to the carrier more firmly than the opening section and is predetermined to remain on the carrier when the opening section is detached.

17. A method for manufacturing a delivery system for percutaneous transluminal angioplasty, the method comprising the steps of:
shaping and/or reshaping a flat carrier with an indentation; and
inserting an angioplasty catheter, which has a hub at a proximal end section and at least one catheter balloon at a distal end section, into the indentation in such a way that the angioplasty catheter is accommodated and held in one or more spiral turns in the indentation.

18. The method according to claim 17, wherein the step of shaping and/or reshaping the flat carrier comprises the step of deep drawing a film or casting a fiber material.

19. The method according to claim 17, further comprising the steps of:
spanning the indentation with a sealing film; and
connecting the sealing film to the flat carrier,
wherein the angioplasty catheter is packaged in a germ-tight manner in a receptacle bounded by the flat carrier and the sealing film.

20. The method according to claim 17, further comprising the steps of:
packing the flat carrier in a closure bag; and
sealing the closure bag,
wherein the flat carrier and the angioplasty catheter held on the flat carrier are packaged in a germ-tight manner in the closure bag.
